(19) United States
(12) Patent Application Publication
(10) Pub. No.: US 2006/0235415 A1
(43) Pub. Date: Oct. 19, 2006

(54) Stainless self-tapping screw

(76) Inventors: To-Ha Loi, Dandenong South (AU); Tran-Tu Loi, Dandenong South (AU)

Correspondence Address: To-Ha Loi, 235 Chung - Ho, Box 8-24, Taipei, TW

(21) Appl. No.: 11/107,719

(22) Filed: Apr. 18, 2005

(51) Int. Cl. A61B 17/58

(52) U.S. Cl. 606/73

(57) ABSTRACT

A stainless self-tapping screw comprises a screw rod made of stainless material; a rear end of the screw rod having an integral formed head and a front end thereof having a polygonal buckling groove; a cutting unit made of a hard material. A front end of the cutting unit having a cutting end and a rear end having an integral formed with a buckling post which is insertable into the buckling groove at a front end of the screw rod. In operation, the cutting unit inserted at a front end of the screw rod serves to drill a hole; then the screw rod is screwed into an metal object; the cutting unit can be taken down as the stainless self-tapping screw is screwed into the metal object.

### FIELD OF THE INVENTION

The present invention relates to screws, and in particular to a stainless self-tapping screw, wherein a cutting unit inserted at a front end of a screw rod makes the stainless self-tapping screw with lower hardness so as to have the effect of a self-tapping screw. Furthermore, when the screw rod is screwed into a metal object. The cutting unit can be taken down to avoid the cutting unit to rust and thus it can retain with a beautiful outlook. When the screw rod is screwed into a metal object, the cutting unit can be taken down to avoid the protrusion of the cutting unit. Thereby a safety use is provided and moreover the cutting unit can be reused.

### BACKGROUND OF THE INVENTION

When it is desired to screw a stud to a metal object, a cutting means is used to drill a hole on the metal object. Thereby the screw can be screwed into the stud for screwing operation. Thereby the operation is inconvenient and labor-wasted.

To resolve the above defect, see FIG. 1, an improvement of the self-tapping screw is disclosed. A rear end of a screw rod 10 has a head 11. A front end of the screw rod 10 has a cutting unit 12. Thereby, to screw the self-tapping screw to a metal object, the cutting unit 12 at the front end of the screw rod 10 will drill a hole. The self-tapping screw will screw into the metal object with the screw rod 10.

However the prior art has improved the defects in the prior art so that the stud can be screwed into an object with less time and force. However this kind of prior art still has the following defects.

The cutting unit 12 has sufficient cutting ability and is integrally formed to a front end of the screw rod 10. The screw rod 10 with a self-tapping stud must be made of metal with great hardness. However the current such kind metal easily rusts so as to affect the appearance of the metal object and the use of the object is confined. Thereby it can be used to a stainless stud with lower hardness. Namely, there is no stainless self-tapping screw being used currently.

Since the cutting unit of the self-tapping stud is integrally formed to a front end of the screw rod 10 so as to prolong the length of the screw rod 10, it is easy to protrude from a surface of an object so as to harm the user.

Furthermore, since the cutting unit of the self-tapping stud is integrally formed to a front end of the screw rod 10, the cutting unit 12 can be used repeatedly. Thus, the cost is high.

### SUMMARY OF THE INVENTION

Accordingly, the primary object of the present invention is to provide a stainless self-tapping screw, wherein since the cutting unit inserted at a front end of the screw rod makes the stainless self-tapping screw with lower hardness has the effect of a self-tapping screw. Furthermore, when the screw rod is screwed into a metal object. The cutting unit can be taken down so as to avoid the cutting unit to rust and thus it can retain with a beautiful outlook. When the screw rod is screwed into a metal object, the cutting unit can be taken down to avoid the protrusion of the cutting unit. Thereby a safety use is provided and moreover the cutting unit can be reused. Thus, the present invention is a safe, cost-saved and convenient screws.

To achieve above object, the present invention provides a stainless self-tapping screw comprises a screw rod made of stainless material; a rear end of the screw rod having an integral formed head and a front end thereof having a polygonal buckling groove; a cutting unit made of a hard material. A front end of the cutting unit having a cutting end and a rear end having an integral formed with a buckling post which is insertable into the buckling groove at a front end of the screw rod. In operation, the cutting unit inserted at a front end of the screw rod serves to drill a hole; then the screw rod is screwed into an metal object; when the stainless self-tapping screw is screwed into the metal object; then the cutting unit can be taken down. The buckling groove is a polygonal groove and the buckling post is a polygonal post.

The various objects and advantages of the present invention will be more readily understood from the following detailed description when read in conjunction with the appended drawing.

### BRIEF DESCRIPTION OF THE DRAWINGS

FIG. 1 shows a prior art self-tapping screw.

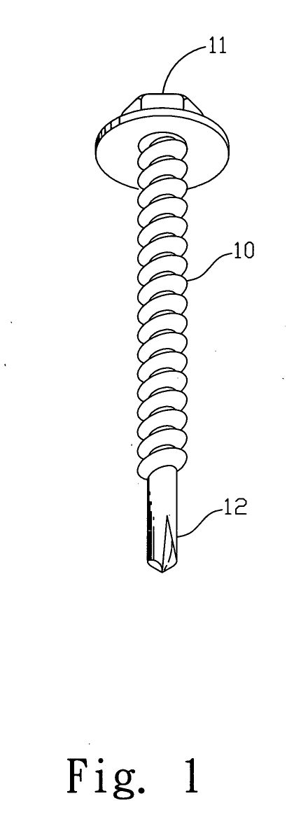

FIG. 2 is an exploded perspective view of the present invention.

FIG. 3 is a plan view of the present invention, wherein it is illustrated that a cutting unit serves to drill a hole in a metal object.

FIG. 4 is a lateral plane view of the present invention, wherein it is illustrated that the cutting unit can be taken down after the present invention is used.

### DETAILED DESCRIPTION OF THE INVENTION

In order that those skilled in the art can further understand the present invention, a description will be described in the following in details. However, these descriptions and the appended drawings are only used to cause those skilled in the art to understand the objects, features, and characteristics of the present invention, but not to be used to confine the scope and spirit of the present invention defined in the appended claims.

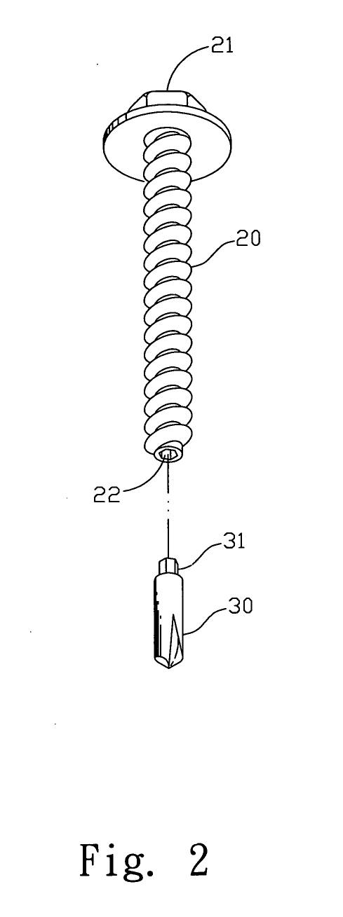

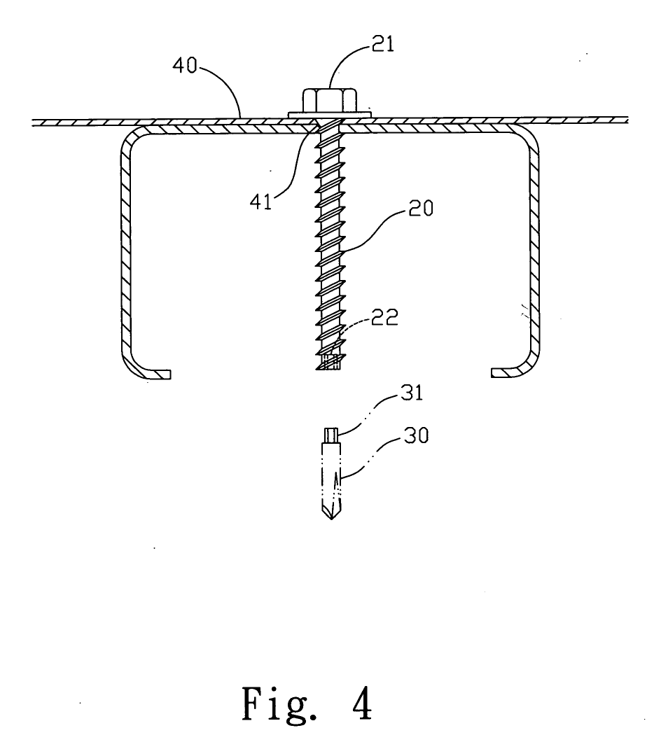

Referring to FIGS. 2 and 4, the stainless self-tapping screw of the present invention is illustrated. The stainless self-tapping screw has the following elements.

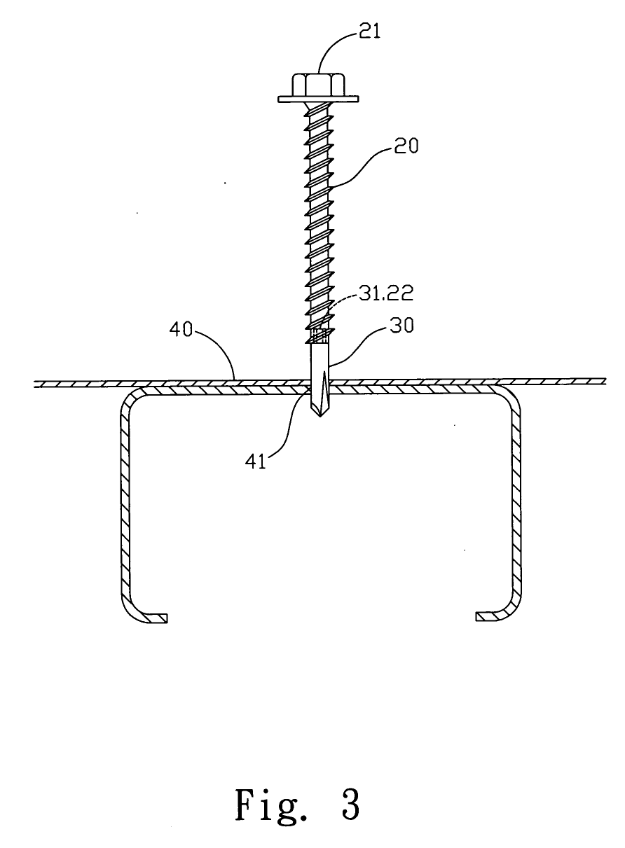

A screw rod 20 (referring to FIG. 3) is made of stainless material. A rear end of the screw rod 20 has an integral formed head 21 and a front end thereof has a polygonal buckling groove 22.

A cutting unit 30 (see FIG. 3) is made of a hard material. A front end of the cutting unit 30 has a cutting end and a rear end has an integral formed with a buckling post 31 which is insertable into the buckling groove 22 at the front end of the screw rod 20. Furthermore, the buckling groove at the front end of the screw rod 20 is a polygonal groove, and the buckling post at the rear end of the screw rod is a polygonal post.

In operation of the present invention, the cutting unit 30 inserted at the front end of the screw rod 20 serves to drill a hole 41 (referring to FIG. 3). Then the screw rod 20 can be screwed into a metal object. Furthermore, when the stainless self-tapping screw of the present invention is screwed into the metal object, the cutting unit 30 can be taken down (referring to FIG. 3).

Advantages of the present invention will be described in the following. Since the cutting unit 30 inserted at a front end of the screw rod 20 makes the stainless self-tapping screw with lower hardness has the effect of a self-tapping screw. Furthermore, when the screw rod 20 is screwed into a metal object 40. The cutting unit 30 can be taken down so as to avoid the cutting unit 30 to rust and thus it can retain with a beautiful outlook. When the screw rod 20 is screwed into a metal object, the cutting unit 30 can be taken down to avoid the protrusion of the cutting unit 30. Thereby a safety use is provided and moreover the cutting unit 30 can be reused. Thus, the present invention is a safe, cost-saved and convenient screws. The present invention is thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A stainless self-tapping screw comprising:
a screw rod made of stainless material; a rear end of the screw rod having an integral formed head and a front end thereof having a polygonal buckling groove;
a cutting unit made of a hard material; a front end of the cutting unit having a cutting end and a rear end having an integral formed with a buckling post which is insertable into the buckling groove at a front end of the screw rod;
wherein in operation, the cutting unit inserted at a front end of the screw rod serves to drill a hole; then the screw rod is screwed into an metal object; the cutting unit can be taken down as the stainless self-tapping screw is screwed into the metal object.

2. The stainless self-tapping screw as claimed in claim 1, wherein the buckling groove at the front end of the screw rod is a polygonal groove.

3. The stainless self-tapping screw as claimed in claim 1, wherein the buckling post at the rear end of the screw rod is a polygonal post.
